(19) United States
(12) Patent Application Publication
(10) Pub. No.: US 2021/0213406 A1
(43) Pub. Date: Jul. 15, 2021

(54) Method and Apparatus for Curing Plant-Based Extracts

(71) Applicants: Adam CHASE, Eugene, OR (US); Brice SHERMAN, Eugene, OR (US); Paul SHERMAN, Springfield, OR (US)

(72) Inventors: Adam CHASE, Eugene, OR (US); Brice SHERMAN, Eugene, OR (US); Paul SHERMAN, Springfield, OR (US)

(73) Assignee: Willamette Valley Alchemy, Eugene, OR (US)

(21) Appl. No.: 16/739,036

(22) Filed: Jan. 9, 2020

(51) Int. Cl. B01J 3/04; C07C 37/56; C07D 311/80; B01D 11/02

(52) U.S. Cl. CPC B01J 3/046; C07C 37/56; C07C 2601/16; B01D 11/0207; B01D 11/0292; C07D 311/80

(57) ABSTRACT

Equipment and processes for curing and decarboxylating botanical oils, and in particular oils such as cannabidiol (“CBD”) and tetrahydrocannabinol (“THC”) from plants of the genus Cannabis (including both THC-lacking industrial hemp and THC-bearing varieties) are described. Lower temperatures, extended cure cycles and inert-gas processing improve product quality and reduce undesired oxidation, resulting in clear, homogenous oils with less tendency to crystallize.

### FIELD

The invention relates to processes and apparatus for purifying essential oils from plant and botanical base stocks.

### BACKGROUND

Many plants produce or develop materials having commercial value as a natural part of their lifecycle. Sometimes, the plants (or parts thereof) can be used as-is: for example, the cotton plant (Gossypium hirsutum and similar species) grow a soft, fluffy staple fiber in a boll, or protective case, that forms around the seeds of the plant. This fiber can be extracted, cleaned and spun into thread or yarn to make various textiles.

The leaves, stems, roots, flowers, fruits or other parts of many plants can also be used more-or-less as-is. For example, apples (genus Malus), oranges (genus Citrus) and bananas (genus Musa) produce fruit that can be eaten directly off the plant; while the leaves of many different species can be steeped in hot water to make tea, and the flowers of hops (genus Humulus) are often used to flavor beer.

Other plants produce materials requiring more extensive processing to place in a convenient form for human or animal use. Sometimes the processing is as simple as heat-treating (i.e., cooking): corn (Zea) and potatoes (Solanum) are more palatable when cooked. Further processing of these plants may yield oils, alcohols or other pharmaceutically-active compounds.

When more-sophisticated processing of a plant or plant part is necessary to obtain a desired product, the apparatus and conditions of processing are often important to the yield and quality (i.e., the purity) of the product. Improvements in apparatus and processing techniques may be of significant value in these situations.

### SUMMARY

Embodiments of the invention are apparatus and processing techniques for purifying oils extracted from plant matter using traditional techniques, where the novel processing phases produce a clear, homogenous, refined oil having a reduced tendency to start the process of nucleation, therefore reducing or preventing crystallization of the oil in storage.

### BRIEF DESCRIPTION OF DRAWINGS

FIG. 1 shows a general-perspective view of an apparatus used to conduct the purifying operations.

FIG. 2 is a high-level flow chart explaining where the novel processing according to an embodiment may occur in an overall extraction-through-administration cycle

FIG. 3 provides additional details concerning the processing operations according to an embodiment.

### DETAILED DESCRIPTION

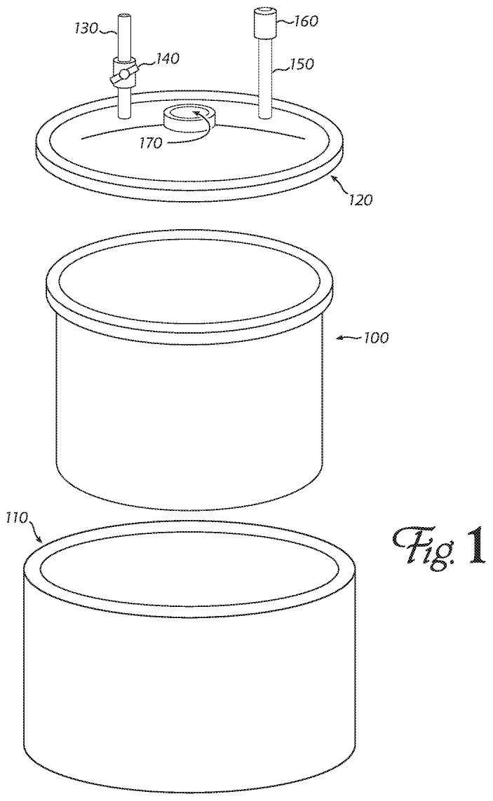

FIG. 1 shows a reaction vessel suitable for conducting a purification process according to an embodiment. A cylindrical vessel 100 having a closed bottom and an open top may be made from an unreactive or low-reactive material such as stainless steel. The vessel is inserted into a suitably sized insulating and heating blanket 110. In a preferred embodiment, the heating blanket is electrically controlled, with a heating capacity at least great enough to achieve a 50°/minute temperature change on the vessel 100 and its contents. A proportional-integral-derivative (“PID”) controller, suitably configured, can achieve a desired target temperature profile with low overshoot or variation.

A lid or cover 120 is secured to the vessel 100 by a tri-clamp system or another suitable fully-sealing mechanism (not shown). The cover includes a first vent 130 having a manual valve 140 and a second vent 150 having an automatic valve 160. The automatic valve may be a blow-off or pressure-limiting valve to prevent intra-chamber pressure from exceeding a particular value, such as a pressure in the range from about 0.5 PSI to about 5.0 PSI above ambient. The automatic valve may seal when intra-chamber pressures are below ambient; to open the vessel when it is in this state, the manual valve can be used to allow an external gas (including without limitation ambient air) back into the vessel. A preferred embodiment may also include a transparent window 170, such as a borosilicate window, through which the vessel contents may be observed.

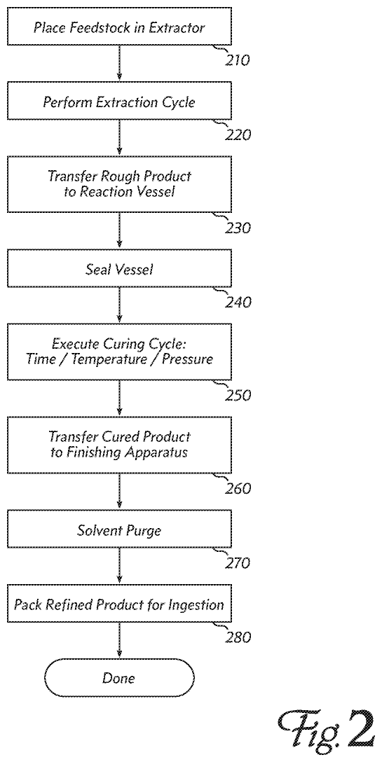

FIG. 2 outlines a complete extraction and refining process that incorporates an embodiment of the invention to improve product quality. First, a vegetable/biomass feedstock is placed in a suitable prior-art extractor (210). The feedstock may be, for example, the flowers, leaves, seeds, stems, roots or multiple parts of one or more plants. These may be fresh, dried, freeze-dried, smoked, frozen, or otherwise processed to impart desired characteristics such as odor or flavor to the extracted product. In one embodiment, the feedstock may be the flowers, leaves, seeds or stems of a member of the genus Cannabis. Oils such as cannabidiol (commonly known by the acronym CBD) and tetrahydrocannabinol (“THC”) can easily be extracted from this stock.

Next, the prior-art extractor is operated (220). Two common extraction techniques that are suitable here are carbon dioxide (CO₂) extraction and hydrocarbon extraction. In CO₂ extraction, the feedstock is exposed to supercritical (liquid) CO₂. In hydrocarbon extraction, the feedstock is exposed to a light hydrocarbon such as butane or propane. In either case, the desired oil dissolves into the solvent (CO₂ or hydrocarbon) at the reaction temperature and pressure. For Hemp CBD oil, hydrocarbon extraction is more efficient (achieves higher yield, in shorter cycle times) than CO₂ extraction, and may be conducted at lower temperatures and pressures. Thus, it is a preferred extraction method.

At the end of the extraction cycle, the oil-bearing solvent is transferred to a reaction vessel for processing according to an embodiment of the invention (230). The reaction vessel is sealed (240) and a curing cycle comprising a predetermined sequence of temperature and pressure sets for predetermined hold times is performed (250). At the end of the curing process, the product (which is in liquid form, but may still include some solvent) is transferred to a finishing station (260). In some implementations, the finishing station is a shallow tray, such as a Pyrex tray. A shallow tray exposes a large surface area of the cured liquid for a given volume thereof. The finishing station (e.g. shallow tray) is placed in a vacuum oven and the pressure is lowered to cause any solvent remaining in the cured product to evaporate (270).

After the finishing (solvent purge) phase, the refined and cured botanical oil can be packed for distribution or use. In one embodiment, the refined product may be packed for ingestion (280), for example by being metered into cartridges suitable for use with a vaporizing apparatus (e.g., a “vape pen.”). In another embodiment, the refined product may be incorporated into an edible product such as a chocolate, gummy or jelly candy, hard candy, or baked product, which may be eaten for therapeutic effects.

The effect of the curing cycle is to decarboxylate the oil or essential oil extracted from the feedstock in a controlled slow manner. A decarboxylated oil is more stable—it does not allow the oil to crystallize or nucleate. By performing the curing cycle in a closed vessel (i.e., the sealed reaction vessel), the oil does not oxidize as easily as an uncured oil. Oxidation can cause the oil to turn a darker color, and reduces the product quality. Cured, decarboxylated oil prepared according to an embodiment of the invention is uniform (homogeonous) and light in color—from a light straw color to almost clear—and about the viscosity of a light honey. (Viscosity can be expressed in units of “poise” [“P”]; oils prepared according to an embodiment have a viscosity between about 10,000 cP [“centipoise”] to about 125,000 cP, largely dependent upon the starting feedstock.)

Thus, products such as vaporizer cartridges filled with an oil prepared according to an embodiment have an extended shelf life and perform better when ingested by a user. Furthermore, the decarboxylated oil is less likely to damage the administration apparatus used by the end user. The improvement in product quality and performance justifies the increased processing time and expense of performing the curing operation.

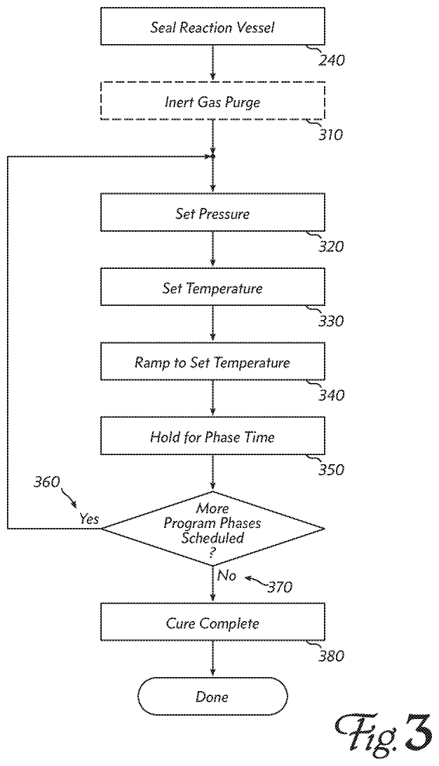

FIG. 3 describes the curing process in greater detail. This starts when the extracted oil (oil and solvent mixture) is placed in the reaction vessel and the vessel is sealed (240). Next—and optionally—the vessel may be purged of ambient atmosphere by reducing the internal pressure using a vacuum pump, then back-filling with an inert gas such as nitrogen or argon, back to an internal pressure set by the blow-off valve. This optional purge-and-backfill step (310) reduces the amount of oxygen in the reaction vessel that could participate in oxidizing reactions during the curing process. Thus, the inert-gas purge may contribute to improvements in product quality.

Next, the vessel pressure may be increased or decreased to a predetermined target pressure (320), although in some processes it is sufficient to provide the reaction chamber with a blow-off valve that simply limits the maximum pressure to, for example, between about 0.5 PSI and about 5.0 PSI over ambient. (It is appreciated that the heating performed in subsequent steps will cause any gas in the reaction vessel to expand, which would increase the internal pressure but for the blow-off valve.)

Now the heating jacket is activated to set the vessel temperature (330). A controller, such as a PID controller, can be used to operate the heating jacket so that it achieves the target temperature quickly, without substantial overshoot, undershoot or variation during the processing phase. In some processing programs, the rate of temperature change may also be specified, so that the controller warms or cools the vessel more slowly than it is capable of doing at full power (a “temperature ramp” (340)). Once the target temperature is reached, the controller holds the temperature steady for a predetermined period of time (the “phase time”) (350).

At the end of the phase time, another time/temperature/pressure phase may be scheduled. If so (360), the next conditions are set (pressure 320, temperature 330) and held for the next phase time (350). If the full curing program has been completed (370), then the product cure is complete (380) and the material in the reaction vessel can be advanced to the next processing step (e.g., solvent purging, 270).

For curing a high-CBD extract, a two-phase program is preferred:

| PHASE | TEMPERATURE | PRESSURE | RAMP | HOLD TIME |
|---|---|---|---|---|
| 1 | 200° F. | +0.5 PSI | * | 48 hours |
| 2 | 145° F. | +0.5 PSI | * | 72 hours+ |

* Ramp times are not critical

For curing a low-CBD extract, a shorter two-phase program is preferred:

| PHASE | TEMPERATURE | PRESSURE | RAMP | HOLD TIME |
|---|---|---|---|---|
| 1 | 190° F. | +2.0 PSI | * | 24 hours |
| 2 | 145° F. | +2.0 PSI | * | 48 hours+ |

Practical temperature control mechanisms, such as a PID controller operating an electrical heating jacket (refer FIG. 1), maintain the apparatus and its contents within a range of, say, ±5° F., ±10° F. or ±25° F. of the target temperature. A narrower range indicates better control of process conditions, but even a wider range may produce acceptable results.

It is appreciated that the decarboxylation reaction can be accomplished in less time, by treating the product at a higher temperature. For example, U.S. Pat. No. 10,143,706 to Kotra et al. discloses performing decarboxylation at temperatures exceeding 100° C. (212° F.)—in many cases, at temperatures substantially in excess of 100° C. The inventors note that the product depicted in Kotra at FIG. 2A is quite dark—indicating that substantial oxidation has occurred.

In the preceding description, numerous details were set forth. It will be apparent, however, to one skilled in the art, that the present invention may be practiced without some of these specific details. In some instances, well-known structures and devices are shown in block diagram form, rather than in detail, in order to avoid obscuring the present invention.

The applications of the present invention have been described largely by reference to specific examples and in terms of particular processing programs. However, those of skill in the art will recognize that extract refinement can also be performed on other botanical-oil/solvent mixtures using the same or similar programs as described here for CBD oil extract, THC oil extract, and related cannabis products from the Cannabis genus. Such variations and alternate feedstocks are understood to be captured according to the following claims.

We claim:

1. A botanical curing system, comprising:
a sealable stainless steel vessel having an automatic pressure-relief exhaust and a manual pressure-relief exhaust; and
a heating jacket having a feedback-governed temperature controller.

2. The botanical curing system of claim 1 wherein the automatic pressure-relief exhaust activates if an internal pressure of the sealable stainless-steel vessel exceeds a predetermined pressure.

3. The botanical curing system of claim 2 wherein the predetermined pressure is between about 0.5 PSI and about 5.0 PSI.

4. The botanical curing system of claim 2 wherein the predetermined pressure is about 0.5 PSI.

5. The botanical curing system of claim 1 wherein the sealable stainless-steel vessel comprises a tri-clamp fitting, and wherein
the sealable stainless-steel vessel further comprises a borosilicate sight glass secured to the tri-clamp fitting.

6. The botanical curing system of claim 1 wherein the feedback-governed temperature controller is provided with a time/temperature profile, and wherein
the heating jacket is controlled by the feedback-governed temperature controller to achieve target temperatures and temperature hold times according to the time/temperature profile.

7. A method of decarboxylating a botanically-derived acid, comprising:
introducing a botanically-derived acid feed stock into a sealable vessel;
sealing the sealable vessel to produce a sealed reaction chamber capable of supporting internal pressures lower than an ambient pressure, and internal pressures up to a predetermined pressure differential above the ambient pressure;
heating the sealed reaction chamber to cause a temperature of the acid feed stock contained therein to approximate a temperature profile over a reaction time; and
after the reaction time has elapsed,
cooling the sealed reaction chamber to an ambient temperature;
unsealing the sealed reaction chamber; and
removing a decarboxylated finished product from the sealable vessel.

8. The method of claim 7, further comprising:
purging the sealable vessel after sealing to replace an ambient gas therein with an inert gas.

9. The method of claim 8 wherein the inert gas is nitrogen or argon.

10. The method of claim 7, wherein the botanically-derived acid feed stock is cannabidiolic acid, and the decarboxylated finished product is cannabidiol.

11. The method of claim 7, wherein the botanically-derived acid feed stock is tetrahydrocannabinolic acid, and the decarboxylated finished product is tetrahydrocannabinol.

12. The method of claim 7, wherein the botanically-derived acid feed stock comprises a solvent.

13. The method of claim 12 wherein the solvent is a light hydrocarbon.

14. The method of claim 7, wherein the reaction time is between about 72 hours and about 120 hours.

15. The method of claim 7, wherein the temperature profile includes a first phase of about 48 hours at a first temperature of about 200° F., followed by a second phase of at least 72 hours at a second temperature of about 145° F., said second temperature lower than said first temperature.

16. The method of claim 7, wherein the temperature profile includes a first phase of about 24 hours at a first temperature of about 190° F., followed by a second phase of at least 48 hours at a second temperature of about 145° F., said second temperature lower than said first temperature.

17. The method of claim 7, further comprising:
processing the decarboxylated finished product at a reduced pressure to evaporate any solvent remaining therein.

18. A method of producing a clear, homogenous botanical oil extract, comprising:
placing frozen material from a Cannabis plant in an extraction apparatus;
exposing the frozen material to a light hydrocarbon solvent to dissolve oils from the frozen material into the solvent and create an oil-bearing solvent;
transferring the oil-bearing solvent to a sealable curing apparatus;
sealing the sealable curing apparatus;
curing the oil-bearing solvent in the sealable curing apparatus by holding a temperature of the sealable curing apparatus at about 190° F. for about 24 hours, then at about 145° F. for at least 48 hours, to produce a cured oil-bearing solvent;
transferring the cured oil-bearing solvent to a solvent-purge apparatus; and
exposing the cured oil-bearing solvent to a reduced pressure to evaporate the solvent, leaving behind the clear, homogenous botanical oil extract.

19. The method of claim 17, wherein the clear, homogenous botanical oil extract is a light color between clear and light straw.

20. The method of claim 17, wherein the clear, homogenous botanical oil extract has a viscosity between about 10,000 cP and about 125,000 cP.
